(19) United States
(12) Patent Application Publication
(10) Pub. No.: US 2011/0263971 A1
(43) Pub. Date: Oct. 27, 2011

(54) Reconfigurable Navigated Surgical Tool Tracker

(76) Inventors: Constantinos Nikou, Monroeville, PA (US); Adam Hahn, Pittsburg, PA (US); Benjamin McCandless, Pittsburgh, PA (US); Steve Hein, Madison, WI (US)

(21) Appl. No.: 13/092,486

(22) Filed: Apr. 22, 2011

Related U.S. Application Data
(60) Provisional application No. 61/342,996, filed on Apr. 22, 2010.

(51) Int. Cl. A61B 5/05

(52) U.S. Cl. 600/424

(57) ABSTRACT

A reconfigurable tracking arrangement for an object includes a repositionable mounting arrangement that may be configured to releasably connect to an object for selectively positioning the same. The mounting arrangement may further include a first piece attached to the object and a second piece that can be repositioned relative to the first piece. In addition, the mounting arrangement may be configured to be secured in at least two stable pre-configured positions about the object.

### CROSS REFERENCE TO RELATED APPLICATION

This application claims the benefit of priority of U.S. Provisional Patent Application Ser. No. 61/342,996, filed Apr. 22, 2010, on which this patent application is based and which is incorporated herein by reference in its entirety.

### FIELD OF THE DISCLOSURE

The present disclosure relates generally to methods, processes, apparatus, and systems for adjustable configurations of a tracking arrangement for a navigated surgical tool. In one embodiment, the present invention is implemented to enhance at least one of access, usability, configuration and overall trackable envelope of the tool.

### BACKGROUND

Optical navigation, as well as other navigation, is used in surgery to track a rigid body's location in space in relation to a tool. These systems often rely upon the use of a camera and markers, the positions of which are tracked by the camera as discuss further hereinbelow. Accordingly, using a known spacial relationship of the markers on the image frame, the 3D position of the tool in relation to the rigid body can be known while the camera can sense the markers. Display software may further be used to display the 3D position of the tool in relation the rigid body so that a virtual, real-time image of the tool and the surrounding anatomy of the patient may be made available to the surgeon to aid in the surgery.

Further, multiple tools and/or objects may be tracked, including rigid patient anatomy such as a bone, in the same workspace with the same camera. However, each object must have its own tracker and the configuration of the markers must be unique for each tool and/or object so as to enable the software (or any other computer processor analyzing image data) to distinguish between tracked objects based on their respective trackers. These tracker frames may be large relative to the tracked objects and anatomy they are used to track and may cause interference with one another.

Since the navigation system is optical, all trackers must remain within the sensing range of the camera during a surgical procedure to avoid complications. If a tracker is physically blocked or moves out of camera's view, the real-time tracking will stop until the tracker is moved back into the workspace sensing range (i.e., back into the camera's view). This creates problems during surgery if the tracked tool and/or objects are not detected and may require repositioning where the tracker is not visible to the camera.

Furthermore, as stated above, one of the requirements for the tool to be trackable is that the tool generally has to be rigid and have a frame rigidly attached to it to ensure spacial integrity. This is required so that the software may infer the location of the tool relative to its frame. The tracker software only “knows” the location of the frame. But, if the software is provided with information that the tool has a given size and shape (e.g., the surgical drill) and is attached to this frame in a specific configuration, then the software “knows” the location of that tool as well. Thus, the location of the tool is intertwined with the location of the tracker. If the tool somehow moves relative to the tracker (e.g., when the tracker mount loosens and the tracker frame shifts relative to the tool), the tool location data from the navigation system is no longer accurate.

Hence, motion of the tool relative to the tracker frame may have to be restricted in order to correctly track the tool's location during surgery. This could create a problem in a variety of surgeries using navigation in that the presence of a tracker and/or navigation instrumentation may prohibit motion of the tool beyond what is “allowed” in the presence of the tracker frame. Such a restriction could create a physical barrier (e.g., when the tracker frame hits something that keeps the surgeon from moving the tracked tool to the proper orientation), or it could create a tracking problem when the tracker frame is blocked from (camera's) view (e.g., when the tool cutting guard prevents a particular cut and the tool must be rotated out of the field of view) and the system cannot locate the tracked tool even when the tool itself may be oriented in an ideal (desired) position according to the surgeon.

For example, in case of a knee replacement procedure, there are portions of the knee (that needed to be cut) that may require that the surgical tool be used in an orientation that would block the tracker frame from being seen by the camera (i.e. be outside of the sensing envelope of the camera). More specifically, a user/surgeon using the tool, rather than holding the tool above the bone and cutting down the bone from above, may need to hold it below the bone and the bone may have to be cut up from below. In order to do this, the tool itself may have to be flipped 180 degrees from its above-the-bone orientation. If the tracker frame is rigidly attached to the top of the tool and visible only in an upright position, then when the tool is flipped 180 degrees, the tracker may no longer be visible to the camera and the tracking may be interrupted, making navigation-based completion of the surgical procedure nearly impossible. Hence, real time tracking of the tool would be interrupted, despite surgically correct (and desirable) location of the tool.

One attempt to address the above problem of restrictions on a (tracked) tool's motion has been to rigidly attach a second tracker frame to the opposite side of the tool so that when the tool is flipped, the second tracker would be visible (to the camera) and tracking of the tool could continue uninterrupted. However, in various applications, such additional tracker may not be desirable or practical. For example, in a surgical bone-cutting procedure, the additional tracker may become cumbersome, add weight, reduce ergonomics, and prohibit gripping of the tool in certain orientations that are required to properly cut the bone.

### SUMMARY OF INVENTION

Therefore, it is an object of the present invention to provide a reconfigurable tool and tracker that overcome some or all of the known drawbacks and deficiencies.

Preferably, the present invention provides methods and devices to reconfigure the object being tracked and/or tracker while retaining its trackability using a single tracker frame. The object being tracked, and/or tracker, may thus be reconfigured to allow for adjustment to various, but still known and rigid configurations relative to the attached tracker frame. As long as the operative software knows that the tool geometry has changed, and is aware of the new geometry (vis-à-vis location of the tracker frame), adjustments to the tool may be made while still retaining tool's trackability. There could be many different reasons for such adjustments. For example, it may be desirable to increase the visible range of the tracked object or tool (e.g., by moving the tracker frame to keep the object in the optical envelope of the navigation system), or to alter the geometry of the tool to increase the access or utility of the tool (e.g. by reconfiguring the tool to accept the tracker at different locations). Hence, the teachings according to the present disclosure could also be used to reduce the number of tools that are tracked since the tracker may be reconfigured into a known position to allow for additional functionality of the object being tracked. For example, if for a certain surgical procedure, two similar tools of different geometry are needed at different stages of the procedure having trackers affixed and verified in a particular fixed tracker orientation, these tools potentially could be combined into one “tool” as discussed later hereinbelow.

In one embodiment, the present disclosure relates to reconfigurable navigated surgical tools and/or trackers that may be adjusted to multiple rigid geometries. In another embodiment, the present disclosure relates to methods and devices to account for reconfiguration and communicate the same to the tracking/navigation software. For example, in one embodiment, a swivel-based tracker mounting mechanism may be provided for use during a knee surgery involving portions of the bone that need to be cut holding the bone-cutting tool upside down while retaining trackability.

In one preferred and non-limiting embodiment, the present invention is directed to a tracking arrangement having a repositionable mounting arrangement that may be configured to releasably connect to an object for selective positioning about the same. The mounting arrangement may further include a first piece attached to the object and as second piece that can be repositioned relative to the first piece. The mounting arrangement may further be configured to be secured in at least two stable pre-configured positions about the object.

In accordance with the present invention, provided is a tracking arrangement including a repositionable mounting arrangement secured to an object and having attachment points configured to receive a frame in at least two stable pre-configured positions about the object.

Further in accordance with the present invention, provided is a navigated surgical tool having attachment points configured to secure a frame in at least two stable pre-configured positions about the object.

These and other features and characteristics of the present invention, as well as the methods of operation and functions of the related elements of structures and combinations of parts and economies of manufacture, will become more apparent upon consideration of the following description and the appended claims with reference to the accompanying drawings, all of which form a part of this specification, wherein like reference numerals designate corresponding parts in the various figs. It is to be expressly understood, however, that the drawings are for the purpose of illustration and description only and are not intended to unduly limit the present invention. As used in the specification and the claims, the singular form of “a”, “an”, and “the” include plural referents unless the context clearly dictates otherwise.

### BRIEF DESCRIPTION OF DRAWINGS

FIG. 1 is a simplified view of an exemplary optical surgical navigation setup during a standard knee replacement surgical procedure;

FIG. 2 is a plan view of an exemplary virtual interface depicting the tracked object in geometrical relationship while a bone of a patient is being resectioned;

FIG. 3 is a perspective view of an exemplary tracker attached to an object for optical surgical navigation;

FIG. 4 is a partial perspective view of an embodiment of a reconfigurable tracker mounted on an object in accordance with the present invention;

FIG. 5 is a partial perspective view of an embodiment of a reconfigurable tracker mounted on an object in accordance with the present invention;

FIG. 6 is a perspective view of an embodiment of a reconfigurable tracker in accordance with the present invention;

FIG. 7 is a partial perspective view of an embodiment of a reconfigurable tracker mounted on an object, and showing other configurations in shadow, in accordance with the present invention;

FIG. 8A is a perspective view of an embodiment of a reconfigurable tracker mounted on an object in accordance with the present invention; and

FIG. 8B is an exploded view of an embodiment of a reconfigurable tracker in accordance with the present invention.

### DETAILED DESCRIPTION OF THE PRESENT INVENTION

For purposes of the description hereinafter, the terms “upper”, “lower”, “right”, “left”, “vertical”, “horizontal”, “top”, “bottom”, “lateral”, “longitudinal” and related derivatives thereof shall relate to the invention as it is oriented in the drawings. However, it is to be understood that the present invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as unduly limiting.

It is to be understood that the invention may assume various alternative variations and step sequences, except where expressly specified to the contrary. It is also to be understood that the specific devices and processes illustrated in the attached drawings, and described in the following specification, are simply exemplary embodiments of the invention.

FIG. 1 illustrates a simplified view of an exemplary optical surgical navigation setup. An infrared camera 10 and a tracker 12 may be used to perform surgical navigation. The tracker 12 may be rigidly attached to any object 14 (e.g., a surgical drill) that the user wishes to track during the surgical procedure. The tracker 12 may include a configuration of one or more IR reflective markers 22 mounted on a frame 20. For example, FIG. 3 illustrates an exemplary tracker 12 including a tracker frame 20 and markers 22. The tracker frame 20 is shown rigidly attached to a rigid object 14 to be tracked by mounting to the rigid object 14 directly.

The camera 10 may thus take continuous images of the workspace during the surgical procedure, and the markers 22 are detected from those pictures. Using the known rigid spatial relationship of the markers 22 on the image frame, the position of the object 14 in a 3D (three dimensional) space may be determined. This object 14 location may be continuously output to a computer program that may integrate this location with patient anatomy as determined from a CT scan and/or ultrasound image, for example. The object 14 location relative to the patient anatomy, such as a bone 18, also may be continuously displayed on a display terminal or monitor 16. Thus, the user/surgeon can visualize and know the location of the object 14 relative to the bone 18 of the patient undergoing the surgical procedure.

It is observed here that the object 14 that is tracked may be various necessary surgical items as long as it has a known physical configuration and the frame 20 of the tracker 12 can be rigidly attached to it in a known configuration. Accordingly, the object 14 may be a surgical drill, a pointer probe, a cutting jig, etc.

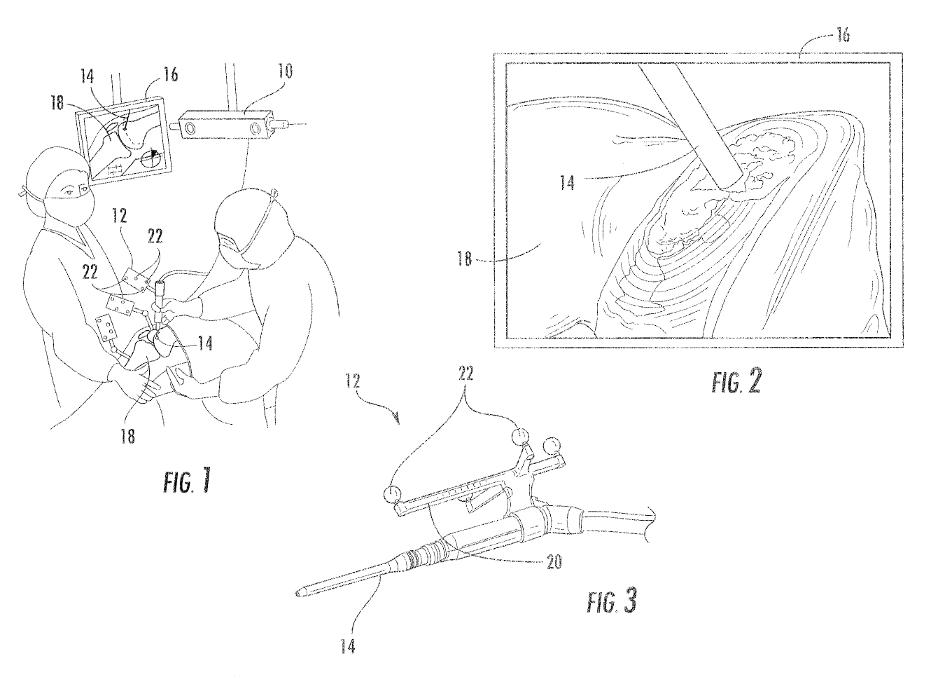

As can be seen in FIG. 2, an exemplary virtual interface depicting the tracked object 14 in geometrical relationship with a bone 18 of a patient is shown. Display software may be used to display the geometry of the object 14 on the display screen 16 so that a virtual, real-time image of the object 14 and the surrounding anatomy of the patient undergoing the surgical procedure may be made available to the surgeon to aid in the surgery. This interface image may remain visible to the surgeon on the display screen 16 during a surgical procedure to assist the user/surgeon.

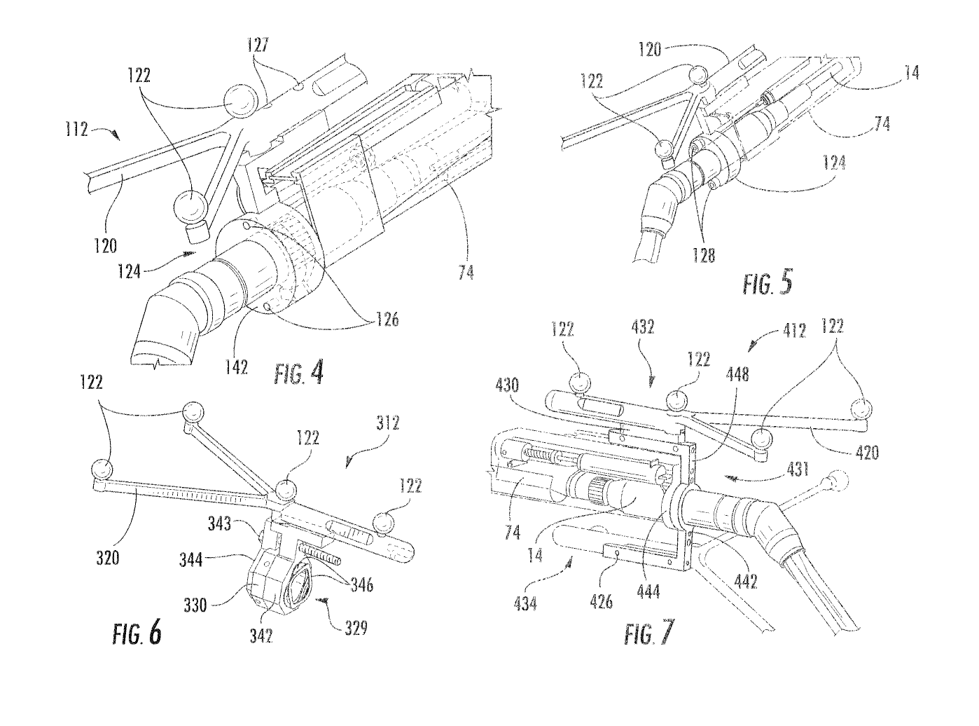

In one preferred and non-limiting embodiment, a reconfigurable tracker 112 may include a frame 120 and markers 122 that may be adjustably installed on a swivel mount 124 as illustrated in FIG. 4. The swivel mount 124 may include ring 142 having apertures 126 that align with apertures (not shown) on housing 74 of the object 14. Accordingly, as can be seen in FIG. 5, the tracker 112 may be secured in a known position to housing 74 by one or more screws 128. However, while screws 128 are removed from apertures 126, the ring 142 may be rotated and thus frame 120 with it. Although two apertures 126 are shown in the illustrated embodiments, it is understood that additional apertures may be provided at other desired angular rotations such that the location of the tracked object 14 can be known.

As can be seen in FIGS. 4-5, if there were a gap between the ring 142 of the swivel mount 124 and the housing 74 positioned on the object 14, any such play permitting the tracker 112 to move slightly even when fully secured to the swivel mount 124 could be resolved by securing screws 128. Accordingly, the collar 140 of the swivel mount 124 may be more securely attached to the housing 74 of the object 14 by tightening screws 128 as illustrated in FIG. 5. Further, in an alternative embodiment, the ring 142 may also be secured in a desired location with retainers 145 such as, for example, ball plungers, positioned in the object 14 and biased toward receiving apertures (not shown) in the inner ring 142 for rigid attachment of the tracker 112 in different known and desired positions.

As can be seen in more detail in FIG. 8A, a swivel mount 184 may have a collar 140 that may be mounted to the tracked object 14. The swivel mount 184 may also include a ring 142 that is configured to rotate about the object 14 and may have an extension 148 to which the tracker 112 may be attached. Extension 148 may be mounted to the ring 142 by one or more attachment members 143 such as screws, bolts, pins, etc., for example, and frame 120 may be mounted to extension 148 by one or more attachment members 123 such as screws, bolts, pins, etc., for example, positioned through apertures 127. Although swivel mount 184 is shown as having at least three components, i.e. collar 140, ring 142, extension 148, it is understood that the swivel mount 184 may be of a unitary construction to within the spirit and scope of the invention. Further, tracker 112 may be of a unitary construction with swivel mount 184 in alternate embodiments of the present invention. Accordingly, when the object 14 is needed to be held upside down to make a cut, the tracker 112 may be swiveled/rotated a desired amount (e.g. 180-degrees) to accompany object 14 rotation and remain in view of the camera 10 in a known spacial configuration.

Further, in order to ensure a certain rotation has be accomplished, rigid stops 121 on the collar 140 may define limits at the ends of the travel of the extension 148 that frame 20 is mounted upon. Once the extension 148 has been positioned within at least one stop 121, such extension 148 may be secured in place with retainers 125 such as, for example, screws, bolts, ball plungers, etc., that may engage recesses and/or apertures (not shown) on extension 148. Accordingly, the tracker 112 may be secured in a known and rigid desired location. As mentioned before, the rotation of the ring 142 may be secured in one of two positions (e.g. 180 degrees apart) at rigid stops 121 within the collar 140 that is attached to the object 14.

In an alternate embodiment, or in addition to other embodiments, and as can be seen in FIG. 8A, the ring 142 may have a secondary extension 144 with apertures 147 to receive ball plungers 145. Ball plungers 145 may be placed in known positions about the body of the object 14 to allow for determination of the position of tracker 112 and thus allow for more variability in optimizing the location of the markers 122 for enabling their detection by the camera 10.

It is observed here that the swivel mount 124 in the embodiments of FIGS. 4-5 allows the tracker 112 to be rotated with precision such as, for example, 180-degrees. Thus, when a user/surgeon switches between cutting bones 18, such as the femur and tibia bones for example, the tracker 112 may desirably be rotated out of the way a known rotational distance to the other side of the object 14. In this manner, the object 14 remains trackable even if held upside-down. Further, rotational indicia 190 may be provided about the object 14 so that the position of extension 148 may be measured.

In another embodiment, an indexable swivel mount 329 may be used as illustrated in FIGS. 6 and 8B. The indexed swivel mount 329 is illustrated having a ring 342 with integral extension 348 for attaching to a tracker 312 via screws 323. As can be seen in FIG. 6, the tracker 312 may further be integrally attached to extension 348 according to one embodiment. The tracker 312 may have, as in other embodiments, markers 122 attached to a tracker frame 320. The indexed swivel mount 329 may further have mechanical indexes 341 for mating with one or more recessed stops 331 on an index receiving collar 330 at known rotational positions. Such mechanical indexes 341 on the ring 342 may be used at various angles about the axis of the swivel mount 329 to assist in assuring a known rotation of the tracker 312 has been made.

In one embodiment, indexes 341 for ridges on the ring 342 that may mate with one or more corresponding recessed stops 331 on collar 330 to provide accurate positioning. Such indexed swivel mount 329 positions may thus allow complete 360-degree motion of the tracker 312 in known increments, such as for example 90-degree increments, using the mechanical indexes 341 resting in recessed stops 331.

The swivel mount 329 may further include a clamp 344 that may be used to prevent accidental motion of the tracker 312 and to reduce the impact of external forces on the orientation/location of the tracker 312. The clamp 344 may be engaged using a thumbscrew 343 to attach the swivel mount 329 to the housing 74. Further, one or more wave spring washers 346 also may be used to provide additional rigidity in coupling with the housing 74.

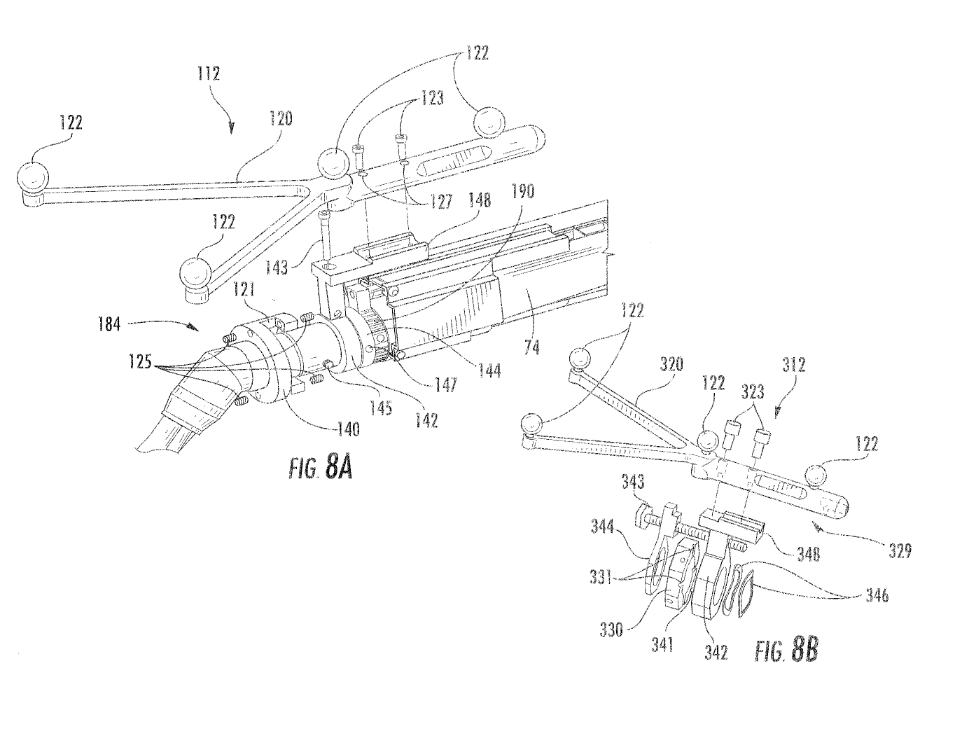

FIG. 7 depicts a snap-lock tracking mount 431 having extension 448, ring portion 442 and secondary extension 444. Secondary extension 444 may be secured to housing 74 and/or the object 14 while ring portion 442 may be secured secondary extension 444 and/or the object 14. Accordingly, the snap tracking mount 431 may be reconfigured to position the tracker 412 in at least two positions 432, 434 such as those illustrated. The tracker 412 may have markers 122 positioned on the frame 420 along with a mount plate 430 connected thereto.

The mounting plate 430 of the snap tracking mount 431 may have attachment points 426 such as apertures, for example, that are positioned into the extension 448 of the tracking mount 431 and snap via friction into a secured known position. Alternatively, mounting plate 430 may be magnetically positioned on extension 448 and/or provided with screws (not shown) to position the tracker 412 via attachment points 426. The snap lock allows quick repositioning of the tracker 412 from the first position 432 to the second position 434. In another embodiment, the extensions 448 may be integral with structure of the object 14 such that the pin-mounting holes 426, or attachment points, may eliminating the need for extension protruding parallel to the object 14.

With reference to the above-discussed mounting arrangements and trackers, after the physical reconfiguration, the software may be made aware of the change to update the display and any other processes that rely on the geometry of the tracked object. There are many different ways to accomplish communication with the software. For example, in one embodiment, the user may explicitly convey (e.g., by proper data entry) the tracked object's new configuration to the software. In another embodiment, a sensor (e.g., a hail effect sensor, an encoder, a proximity sensor, a barcode reader, an RF (radio frequency) ID tag reader, an LVDT (linear variable differential transformer), etc.) may be used to automatically sense the change in tracked object's configuration and feed the most-recent configuration information to the navigation software. In another embodiment, the system software may use the location or orientation of the tracked tool to infer the tool's geometrical configuration. For example, a knee replacement procedure may require work on the anterior to posterior of the femur. If the tracked tool has two configurations (e.g. one for the anterior and one for posterior), the software may infer the configuration the tool is in based on the site currently being cut. Accordingly, the navigation/tracking software may be explicitly “told” by the user/surgeon as to the configuration in which the object 14 is currently positioned. Several configurations may be possible, whether the selected tracker and mounting configuration is in the “original” configuration such as that shown in FIG. 4 or FIG. 5, for example, or the tracker is in an indexed position in case of the swivel mount of FIG. 6, or the tracker is rotated 180-degrees from the “original” position and “snapped” into place as illustrated in FIG. 7.

According to the present invention, there are multiple ways to reconfigure a trackable object or tool (e.g., a surgical drill) into various known rigid positions. For example, in one embodiment discuss herein above, indexable positions (linear or rotational) may be used, whereby an adjustable component (i.e., the tracked tool) snaps into known rigid positions. The indexable part may be the tracker frame itself, or in another embodiment, some part of the tracked object. In another embodiment, the tracked tool may have an infinitely adjustable component with graduated scale. A user may adjust the tool to a known position based on the scale and convey the tool position to the software via user interface. In a further embodiment, the position of the infinitely adjustable component may be monitored by a sensor in communication with the software. In an alternative embodiment, the tracked tool may have swappable geometry. The user could remove a portion of the object and replace it with a different known component. In a still further embodiment, the user may move a component of the tracked tool and reattach the component in a different, but known, location such as is shown in FIG. 7 in shadow for example.

The foregoing describes various approaches for more flexible surgical cutting orientations while retaining a visual line of sight from the camera to the tracker in an optically navigated surgical procedure. Thus, the surgeon may hold a surgical tool in any orientation and, after a possible adjustment, the tool remains trackable. The flexibility provided by the various tracker mounting mechanisms described herein may be useful in various surgical procedures that require the surgeon to hold surgical tools in different orientations due to difficult to reach areas of human anatomy. As mentioned before, there could be many different reasons for adjustments to tool orientation. For example, it may be desirable to increase the visible range of the tracked object or tool (e.g., by moving the tracker frame to keep the object in the optical envelope of the navigation system), or to alter the geometry of the tool to increase the access or utility of the tool and thus provide the functionality of multiple tools via a single tracked tool.

It is noted here that although the foregoing discussion is primarily related to an exemplary navigated surgical tool, the tracker mounting mechanism discussed herein may be applied to any (tracked) tool that uses navigation (e.g. electromagnetic, mechanical, active optical, passive optical, etc.) regardless of whether the tool is surgical in nature or not. For example, in addition to the surgical bone-cutting tool discussed herein, the teachings of the present disclosure may be applied to surgical probes, needles, bones, etc. The trackers may thus be designed to be adjusted to any predetermined and rigid location assuming that the tracking software may be calibrated to the new orientation.

Although the invention has been described in detail for the purpose of illustration based on what is currently considered to be the most practical and preferred embodiment(s), it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment may be combined with one or more features of any other embodiment. Since other modifications and changes varied to fit particular operating requirements and environments will be apparent to those skilled in the art, the invention is not considered limited to the example chosen for purposes of disclosure, and covers all changes and modifications which do not constitute departures from the spirit and scope of this invention.

What is claimed is:

1. A tracking arrangement, comprising:
a repositionable mounting arrangement configured to releasably connect to an object for selective positioning about the object, said repositionable mounting arrangement comprising:
a first piece attached to the object; and
a second piece that can be repositioned relative to said first piece; and
wherein said repositionable mounting arrangement is configured to be secured in at least two stable pre-configured positions about the object.

2. The tracking arrangement of claim 1, wherein said first piece comprises a housing that is configured to receive at least one attachment member for securing said second piece to the housing.

3. The tracking arrangement of claim 1, wherein said first piece further comprises at least one aperture therethrough for receiving an attachment member forconnecting to said repositionable mounting arrangement.

4. The tracking arrangement of claim 1, wherein said second piece comprises at least one extension having at least one aperture therethrough for receiving an attachment member for connecting said at least one extension to said first piece.

5. The tracking arrangement of claim 4, wherein said at least one extension comprises at least two extensions positioned in opposing directions from said first piece.

6. The tracking arrangement of claim 4, wherein said at least one extension comprises apertures for receiving a mounting plate in said at least two stable pre-configured positions.

7. The tracking arrangement of claim 1, wherein said repositionable mounting arrangement further comprises a mounting plate configured to be releasably secured to said second piece.

8. The tracking arrangement of claim 1, wherein said first piece further comprises at least one recess configured to receive a portion of said second piece in one of said at least two stable pre-configured positions.

9. The tracking arrangement of claim 8, wherein said repositionable mounting arrangement further comprises at least one securing member configured to engage one of said first piece and said second piece in said at least two stable pre-configured positions.

10. The tracking arrangement of claim 9, further comprising a clamp having an aperture positioned therethrough for receiving said at least one attachment member and configured to abut said second piece and retain said second piece adjacent said first piece in said at least two stable pre-configured positions.

11. The tracking arrangement of claim 1, further comprising at least one retainer configured to engage one of said first piece and said second piece in said at least two stable pre-configured positions.

12. The tracking arrangement of claim 1, further comprising a frame configured to support at least one marker and configured to connect to said repositionable mounting arrangement.

13. The tracking arrangement of claim 1, further comprising rotational indicia configured to indicate the rotation of said repositionable mounting arrangement.

14. A tracking arrangement, comprising:
a repositionable mounting arrangement secured to an object, the repositionable mounting arrangement comprising attachment points configured to receive a frame in at least two stable pre-configured positions about the object.

15. The tracking arrangement of claim 14, wherein said frame is configured to support at least one marker and is removably connect to said repositionable mounting arrangement.

16. The tracking arrangement of claim 14, wherein said repositionable mounting arrangement further comprises a mounting plate configured to be secured to said frame.

17. The tracking arrangement of claim 14, wherein said repositionable mounting arrangement further comprises an extension having attachment points.

18. The tracking arrangement of claim 14, further comprising a housing affixed to the object and configured to receive at least one attachment member for securing said repositionable mounting arrangement to said housing.

19. A navigated surgical tool comprising:
A tool;
attachment points configured to secure a frame in at least two stable pre-configured positions about said tool; and
wherein said frame is configured to support at least one marker.

20. The navigated surgical tool of claim 19, further comprising a mounting plate configured to releasably attach said frame to said at least two stable pre-configured positions.
